(19) United States
(12) Patent Application Publication
(10) Pub. No.: US 2023/0063738 A1
(43) Pub. Date: Mar. 2, 2023

(54) ANAEROBIC IMMOBILIZED BACTERIAL AGENT, PREPARATION METHOD FOR SAME, AND APPLICATIONS THEREOF

(71) Applicant: TONGJI UNIVERSITY, Shanghai (CN)

(72) Inventors: PINJING HE, Shanghai (CN); HAOWEN DUAN, Shanghai (CN); FAN LYU, Shanghai (CN); HUA ZHANG, Shanghai (CN); LIMING SHAO, Shanghai (CN)

(21) Appl. No.: 17/794,996

(22) PCT Filed: Apr. 15, 2021
(86) PCT No.: PCT/CN2021/087449

§ 371 Date: Jul. 25, 2022

(30) Foreign Application Priority Data
Jul. 15, 2019 (CN) 202020678320.8

(51) Int. Cl. C12P 5/02; C12N 11/084; C12N 1/20

(52) U.S. Cl. CPC C12P 5/023; C12N 1/20; C12N 11/084

(57) ABSTRACT

Provided are an anaerobic immobilized bacterial agent, a preparation method for same, and applications thereof. The preparation method for the bacterial agent is: selecting four different anaerobic functional bacterial strains, utilizing a pure bacteria culturing technique to produce corresponding culture broths, then mixing the four culture broths according to a certain volume ratio to acquire a compound functional broth, subsequently concentrating into a functional flora precipitation, then dissolving the functional flora precipitation into a polyvinyl alcohol aqueous solution, dripping the solution into a first buffer solution to produce polyvinyl alcohol gel beads, and placing the gel beads produced into a second sulfate-containing buffer solution to produce sulfate-modified polyvinyl alcohol gel beads, that is, the anaerobic immobilized bacterial agent.

### FIELD

The present invention belongs to the field of environmental engineering materials, in particular relates to an anaerobic immobilized bacterial agent, a preparation method for same, and applications thereof.

### BACKGROUND

As one of the effective biological treatment technologies, anaerobic digestion can control the environmental pollution of the easily degradable biomass waste, meanwhile can convert the organics contained to energy gas which mainly contains methane. When feed stock is the biomass waste with a high solid content (such as kitchen waste, livestock manure and sewage sludge, etc.), organic acids mainly containing acetic acid are easy to accumulate rapidly in the anaerobic digestion reactor, which can induce the acid inhibition on methanogens, eventually resulting in an unstable methanogenesis and even the failure operation of reactor. Especially, organic acids can be further accumulated when ammonia inhibition occurs simultaneously, therefore the dual inhibition of ammonia and organic acids generates.

To solve the problem of the rapid accumulation of organic acids, bioaugmentation technique by adding external microorganisms into the anaerobic digestion reactor are applied to improve the degradation rate of lipid, consume excess organic acids, shorten the lag period of methanogenesis, and finally restore the normal operation of the reactor. At present, there are four major problems in the bioaugmentation technique: 1. suspended cultivation mode is easy to induce the loss of external functional microbial consortia; 2. external functional microbial consortia die out quickly due to their poor adaption to the physical and chemical conditions in the reactor, or they are outcompeted by the native microorganisms in the reactor; 3. the external functional microbial consortia using a single strain is easy to die out, and the external functional microbial consortia using compound strains is inefficient due to the lack of synergistic metabolism, therefore the inoculation amount or the inoculation frequency needs to be increased; 4. the cultivation of pure anaerobic microorganisms is difficult and their generation time is long, which cannot meet the requirements of full-scale reactors; 5. the external functional microbial consortia are susceptible to high ammonia concentration and cannot solve the problem of organic acid accumulation under ammonia inhibition. In recent years, a series of methods have been developed in China to relieve the organic acid inhibition in anaerobic digestion reactors. For example, novel anaerobic digestion reactors have been designed by Chinese patents with published number CN202089962U, CN110184171A and CN201644487U, but the technique is not suitable for the anaerobic digestion reactors in which organic acid inhibition has occurred. Adding carbon materials has been disclosed by Chinese invention patents with published number CN107475304A, CN109554402A and CN109182390A, but the technique needs to replenish carbon materials constantly to offset the loss of carbon materials, and the leachate of carbon materials also has a potential toxic effect on microorganisms. Adding an ion exchange resin device has been disclosed by Chinese invention patent with published number CN102992478B, but the technique needs to regenerate the ion exchange resin, and also needs to treat the waste liquid during regeneration, which increasing the process cost. The compound enzyme agent which mainly contains liquefaction enzyme, glucoamylase, cellulase and lipase, has been disclosed by Chinese invention patent with published number CN103014070B, and the compound bacterial agent which mainly contains Pelotomaculum schinkii, has been disclosed by Chinese invention patent with published number CN106085926A, the techniques can relieve the organic acid inhibition by promoting microbial metabolism, but enzyme agent and bacterial agent can loss attributing to they are not being immobilized, consequently the effects of compound enzyme agent and compound bacterial agent are weaken. However, the method of relieving anaerobic digestion organic acid inhibition in situ by immobilized bacterial agents has not been reported.

### SUMMARY OF THE DISCLOSURE

To solve the above technical problems, the present invention provides an anaerobic immobilized bacterial agent, a preparation method for same, and applications thereof.

The present invention provides a preparation method of anaerobic immobilized bacterial agent, characterized by comprising the following steps: step 1, culturing four different anaerobic functional bacterial strains respectively at a certain temperature to acquire corresponding culture broths, then mixing the four culture broths according to a certain volume ratio to acquire a compound functional broth; step 2, concentrating the compound functional broth to acquire a functional flora precipitation; step 3, dissolving the functional flora precipitation into a polyvinyl alcohol aqueous solution to acquire a functional flora polyvinyl alcohol aqueous solution, then dripping the functional flora polyvinyl alcohol aqueous solution into a first buffer solution to acquire polyvinyl alcohol gel beads; step 4, placing the polyvinyl alcohol gel beads into a second sulfate-containing buffer solution to acquire sulfate-modified polyvinyl alcohol gel beads, that is, the anaerobic immobilized bacterial agent, and the anaerobic functional bacterial strains are Coprothermobacter proteolyticus, Thermacetogenium phaeum, Methanosarcina barkeri and Methanothermobacter thermautotrophicus.

The preparation method of anaerobic immobilized bacterial agent provided by the present invention, also has following characteristics: the mass percentage of polyvinyl alcohol in the polyvinyl alcohol aqueous solution in step 3 is 10-15%; the volume ratio of the compound functional broth in step 1 to the polyvinyl alcohol aqueous solution in step 3 is 10:1-20:1.

The preparation method of anaerobic immobilized bacterial agent provided by the present invention, also has following characteristics: the first buffer solution per liter contains 0.15-0.2 mol of Na₂HPO₄, 0.2-0.25 mol of NaH₂PO₄ and 50-60 g of H₃BO₃; the second sulfate-containing buffer solution per liter contains 1-1.5 mol of Na₂SO₄; the diameter of the anaerobic immobilized bacterial agent is 0.5-1 cm.

The present invention also provides an anaerobic immobilized bacterial agent made by the preparation method of anaerobic immobilized bacterial agent, the anaerobic immobilized bacterial agent is circular gel beads which are generated by wrapping gel in a thin film, and the diameter of the circular gel beads is 0.5-1 cm.

The present invention also provides an application of the anaerobic immobilized bacterial agent for anaerobic digestion.

The application of the anaerobic immobilized bacterial agent for anaerobic digestion provided by the present invention, also has following characteristics: culturing four anaerobic functional bacterial strains respectively to acquire corresponding culture broths at a certain temperature utilizing a pure bacteria culturing technique, OD600 of the culture broths is 15-20, then mixing different culture broths according to a certain volume ratio to acquire a compound functional broth; specific operation of the anaerobic digestion is that adding the anaerobic immobilized bacterial agent into an anaerobic digestion reactor to anaerobic digesting, the cultivation temperature of the anaerobic functional bacterial strains is the same with the anaerobic digesting temperature of the anaerobic immobilized bacterial agent in the anaerobic digestion reactor; four anaerobic functional bacterial strains are Coprothermobacter proteolyticus, Thermacetogenium phaeum, Methanosarcina barkeri and Methanothermobacter thermautotrophicus respectively.

The application of the anaerobic immobilized bacterial agent for anaerobic digestion provided by the present invention, also has the following characteristic: the minimum dosage of the compound functional broth is calculated by the following formula:

$${V}_{0} = \frac{{C}_{t o t a l} \times {10}^{- \text{pH}} \times {C}_{v z} \times {V}_{1}}{0.0072} ,$$

V₀ is the minimum dosage of the compound functional broth, L; Cₜₒₜₐₗ is the total concentration of organic acids in the anaerobic digestion reactor, in terms of acetate, mM; pH is the pH in the anaerobic digestion reactor; Cᵥₛ is the concentration of volatile suspended solids in the anaerobic digestion reactor, g/L; V₁ is the effective working volume of the anaerobic digestion reactor, L.

The application of the anaerobic immobilized bacterial agent for anaerobic digestion provided by the present invention, also has following characteristics: when the ammonia concentration in the anaerobic digestion reactor is ≤ 4 g/L and the temperature is 30-43° C., the volume ratio of the Coprothermobacter proteolyticus, the Thermacetogenium phaeum, the Methanosarcina barkeri and the Methanothermobacter thermautotrophicus in the compound functional broth is 2-1:3-1:5-1:2-1; when the ammonia concentration in the anaerobic digestion reactor is ≤ 4 g/L and the temperature is 50-65° C., the volume ratio of the Coprothermobacter proteolyticus, the Thermacetogenium phaeum, the Methanosarcina barkeri and the Methanothermobacter thermautotrophicus in the compound functional broth is 3-1:4-1:2-1:4-1.

The application of the anaerobic immobilized bacterial agent for anaerobic digestion provided by the present invention, also has following characteristics: when the ammonia concentration in the anaerobic digestion reactor is 4-7 g/L and the temperature is 30-43° C., the volume ratio of the Coprothermobacter proteolyticus, the Thermacetogenium phaeum, the Methanosarcina barkeri and the Methanothermobacter thermautotrophicus in the compound functional broth is 2-1:2-1:5-1:3-1; when the ammonia concentration in the anaerobic digestion reactor is 4-7 g/L and the temperature is 50-65° C., the volume ratio of the Coprothermobacter proteolyticus, the Thermacetogenium phaeum, the Methanosarcina barkeri and the Methanothermobacter thermautotrophicus in the compound functional broth is 3-1:3-1:4-1:5-1.

The application of the anaerobic immobilized bacterial agent for anaerobic digestion provided by the present invention, also has following characteristics: the anaerobic immobilized bacterial agent can be desiccated to acquire desiccated gel beads, and the desiccated gel beads can be activated for 12-24 h before adding them into the anaerobic digestion reactor, the nutrient solution used for activation is the cultivation solution used for culturing the anaerobic functional bacterial strains when utilizing a pure bacteria culturing technique.

According to the preparation method and the application of anaerobic immobilized bacterial agent provided by the present invention, selecting four different anaerobic functional bacterial strains, and utilizing a pure bacteria culturing technique to acquire corresponding culture broths, then mixing the four culture broths according to a certain volume ratio to acquire a compound functional broth, subsequently concentrating the compound functional broth into a functional flora precipitation, then dissolving the functional flora precipitation into a polyvinyl alcohol aqueous solution, dripping the solution acquired into a first buffer solution to acquire polyvinyl alcohol gel beads, placing the polyvinyl alcohol gel beads into a second sulfate-containing buffer solution for modification, and the sulfate-modified polyvinyl alcohol gel beads are the anaerobic immobilized bacterial agent. The anaerobic immobilized bacterial agent is formed by the gelation of polyvinyl alcohol aqueous solution, and it is circular gel beads which are generated by wrapping gel in a thin film, the diameter of the circular gel beads is 0.5-1 cm. The anaerobic immobilized bacterial agent can be directly added into an anaerobic digestion reactor to anaerobic digestion without modifying the reactor, it can effectively relieve anaerobic digestion organic acid inhibition in situ and also can relieve organic acid accumulation under ammonia inhibition.

Coprothermobacter proteolyticus decomposes cell debris, proteins and lipids rapidly. Thermacetogenium phaeum decomposes acetic acid into carbon dioxide and hydrogen using syntrophic acetate oxidation. Methanothermobacter thermautotrophicus carries out hydrogenotrophic methanogenesis, and consumes hydrogen to create a favorable thermodynamic environment for Thermacetogenium phaeum. Methanosarcina barkeri consumes acetic acid and hydrogen by acetoclastic methanogenesis and hydrogenotrophic methanogenesis respectively. Therefore, the compound microbial community can accelerate the decrease of organic acid loading, and restore the pH of the anaerobic digestion reactor, further relieve the toxic effect of organic acid inhibition on the native microorganisms in the reactor, and finally restore the functional activity of the native microorganisms. Moreover, the adding of the compound microbial community can avoid installing organic acid separation devices, adding buffer materials and modifying the reactors, thus reducing the process cost. In addition, Coprothermobacter proteolyticus, Thermacetogenium phaeum, Methanothermobacter thermautotrophicus and Methanosarcina barkeri are all ammonia-tolerant strains, so the compound functional broth can relieve organic acid accumulation under ammonia inhibition. In conclusion, the compound functional broth is not only acid-tolerant but also ammonia-tolerant, and it has a wide applicability.

The present invention uses sulfate as a modifier, which overcomes the disadvantages where traditional polyvinyl alcohol-boric acid gel beads cannot be desiccated for preserving. The core of sulfate-boric acid has enough structural flexibility and structural strength to support the gel beads, therefore it can maintain the complete structure of the immobilized bacterial agent and the resistance to external impact.

Meanwhile, immobilizing the external compound functional bacterial agent, rather than letting them suspend in reactors in the form of free cells, can allow external bacterial agent to better colonize the native microorganisms in the reactors, and avoid the large-scale loss of external bacterial agent along with effluent, so there is no need to replenish external bacterial agent constantly. And immobilizing the external compound functional bacterial agent can also increase the cell density of the external bacterial agent, improving the treatment effect.

In addition, the modified gel beads can be mass-produced and transported over long distance because they can be desiccated for preserving, they can be used for the anaerobic digestion reactors from different geographical regions. The modified gel beads overcome the disadvantage of the common gel beads which must be prepared near the anaerobic reactors and used immediately, because the common gel beads cannot be desiccated.

It can be known from the above that the preparation method provided by the present invention is simple, low energy consumption, and the immobilized bacterial agent acquired by this method is acid-tolerant and ammonia-tolerant. The immobilized bacterial agent can significantly improve the ability of the microorganisms to decompose the accumulated organic acids in the anaerobic digestion reactor, and there is no need to replenish the bacterial agent constantly. Compared with the prior art, the preparation method provided by the present invention has a stable effect and a low cost, and this method has no need to modify the existing reactors. The preparation method provided by the present invention can solve the problems of the loss of functional bacterial strains and the poor self-resilience of the anaerobic digestion microorganisms, and can relieve organic acid inhibition and organic acid accumulation under ammonia inhibition effectively during the treatment of organic garbage and sewage.

### BRIEF DESCRIPTION OF THE DRAWINGS

FIG. 1 is the preparation flow chart of anaerobic immobilized bacterial agent provided by the present invention.

### DETAILED DESCRIPTION

In order to easy understand the technical methods, creative characteristics, purposes and effect of the prevent invention, an anaerobic immobilized bacterial agent, a preparation method and applications thereof provided by the present invention will be further described in detail below with reference to embodiments and draws.

Unless special explanation, the raw materials and reagents used in the embodiments of the present invention are purchased through general commercial ways.

The anaerobic functional bacterial strains used in the present invention are Coprothermobacter proteolyticus (DSM 5265), Thermacetogenium phaeum (DSM 26808), Methanosarcina barkeri (DSM 800) and Methanothermobacter thermautotrophicus (DSM 1053) respectively, which are all purchased through general commercial ways.

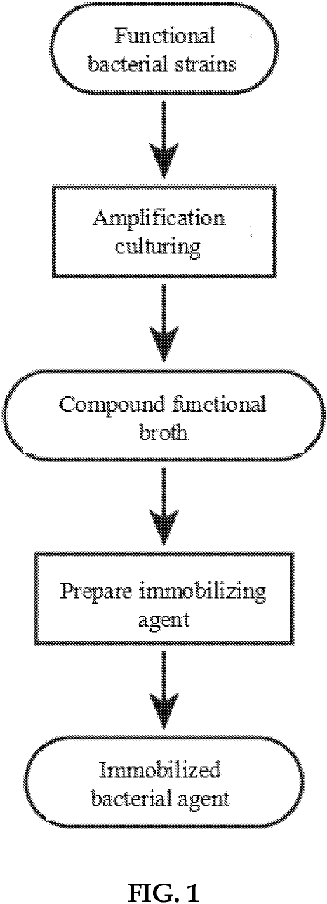

FIG. 1 is the preparation flow chart of anaerobic immobilized bacterial agent provided by the present invention. As shown in FIG. 1, the preparation method of anaerobic immobilized bacterial agent comprises the following steps:
step 1, culturing four different anaerobic functional bacterial strains respectively at a certain temperature to acquire corresponding culture broths, then mixing the four culture broths according to a certain volume ratio to acquire a compound functional broth;
step 2, concentrating by centrifuging the compound functional broth to acquire a functional flora precipitation;
step 3, dissolving the functional flora precipitation into a polyvinyl alcohol aqueous solution to acquire a functional flora polyvinyl alcohol aqueous solution, then dripping the functional flora polyvinyl alcohol aqueous solution into a first buffer solution, standing for 24 h to acquire polyvinyl alcohol gel beads;
step 4, placing the polyvinyl alcohol gel beads into a second sulfate-containing buffer solution, standing for 48 h to acquire sulfate-modified polyvinyl alcohol gel beads, the diameter of which is 0.5-1 cm, that is, the anaerobic immobilized bacterial agent.

In step 1, four anaerobic functional bacterial strains are Coprothermobacter proteolyticus, Thermacetogenium phaeum, Methanosarcina barkeri and Methanothermobacter thermautotrophicus. Using the activated broth of the anaerobic functional bacterial strains to anaerobic culturing the four anaerobic functional bacterial strains respectively utilizing a pure bacteria culturing technique to acquire corresponding culture broths. The redox potential of the culture broths is below -300 mV, OD600 is 15-20, OD600 of the each culture broth is same.

Specific operation of the pure bacteria culturing technique is that inoculating the activated broth into the culture solution in fermenter at anaerobic and aseptic environment, the inoculation amount is 2-5 %, and the stirring speed of the fermenter is 150 r/min. The culture broths were concentrated using sterile polyethersulfone hollow fiber installing at the culture broth outlet of the fermenter, until the OD600 of the broths reach 15-20. The body of the fermenter is made of borosilicate glass with the height is 3.8 L, which was injected with 2 L of culture solution. The inlet and outlet of the fermenter are sealed by 0.22 µm of sterile filter membrane, and the fermenter are sterilized for 20 min at 121° C.

Mixing four culture broths of Coprothermobacter proteolyticus, Thermacetogenium phaeum, Methanosarcina barkeri and Methanothermobacter thermautotrophicus according to a certain volume ratio to acquire a compound functional broth.

The culture solution for culturing anaerobic functional bacterial strains per liter contains 2 g of CH₃COONa, 1 g of NH₄Cl, 0.4 g of K₂HPO₄•3H₂O, 0.2 g of MgCl₂•6H₂O, 0.1 g of CaCl₂•2H₂O, 0.2 g of Na₂ S•9H₂O, 4 mg of FeCl₂•4H₂O, 0.2 mg of H₃BO₃, 1 mg of ZnCl₂, 0.2 mg of CuCl₂•2H₂O, 1 mg of MnCl₂•4H₂O, 0.1 mg of Na₂MoO₄•4H₂O, 1 mg of AlCl₃•6H₂O, 2 mg of CoCl₂·6H₂O, 0.2 mg of NiCl₂•6H₂O, 2 mg of Na₂SeO₃•5H₂O, 10 mg of EDTA-2Na, 0.5 mg of pyridoxine hydrochloride, 0.3 mg of thiamine hydrochloride, 0.3 mg of D-calcium pantothenate, 0.1 mg of folic acid, 0.3 mg of riboflavin, 0.3 mg of nicotinic acid, 0.3 mg of P-aminobenzoic acid, and 0.1 mg of vitamin B₁₂, pH is 6.8-7.0.

In step 3, the mass percentage of polyvinyl alcohol in the polyvinyl alcohol aqueous solution is 10%-15%, the volume ratio of the compound functional broth in step 1 to the polyvinyl alcohol aqueous solution in step 3 is 10:1-20:1.

In step 3, the first buffer solution per liter contains 0.15-0.2 mol of Na₂HPO₄, 0.2-0.25 mol of NaH₂PO₄ and 50-60 g of H₃BO₃, the rest is sterile water.

The second buffer solution per liter contains 1-1.5 mol of Na₂SO₄, the rest is sterile water.

The anaerobic immobilized bacterial agent is formed by the gelation of polyvinyl alcohol aqueous solution, and is circular gel beads which are generated by wrapping gel in a thin film. The diameter of the circular gel beads is 0.5-1 cm, wherein anaerobic functional bacterial strains are wrapped in the gel beads.

In the present invention, the gel beads just prepared can be used directly, also can be desiccated to acquire the desiccated gel beads for convenient transportation and storage. The desiccated gel beads need to be activated before using, named as the first desiccation- activation. The gel beads after the first desiccation- activation also can be conducted the second desiccation-activation, and the activity after the second desiccation- activation maintains at 0-90 %, which providing a stronger using flexibility.

In the embodiments of the present invention, the way of desiccating gel beads is that placing the gel beads in the environment of 80° C., and drying for 24 h to acquire the desiccated gel beads.

Applying the anaerobic immobilized bacterial agent to the anaerobic digestion technology, can relieve organic acid inhibition and the organic acid accumulation under the condition of ammonia inhibition effectively. In the embodiments of the present invention, specific operation of the application is adding the anaerobic immobilized bacterial agent into an anaerobic digestion reactor, the dosage depends on the amount of the compound functional broth in step 1, and the minimum dosage of the compound functional broth is calculated by the following formula:

$${V}_{0} = \frac{{C}_{t o t a l} \times {10}^{- \text{pH}} \times {C}_{v s} \times {V}_{1}}{0.0072}$$

V₀ is the minimum dosage of the compound functional broth, L, Cₜₒₜₐₗ is the total concentration of organic acids in the anaerobic digestion reactor, in terms of acetate, mM, pH is the pH in the anaerobic digestion reactor, Cᵥₛ is the concentration of volatile suspended solids in the anaerobic digestion reactor, g/L, V₁ is the effective working volume of the anaerobic digestion reactor, L.

The cultivation temperature of the anaerobic functional bacterial strains is the same with the anaerobic digesting temperature of the anaerobic immobilized bacterial agent in the anaerobic digestion reactor, namely, is the same with the operating temperature of the anaerobic digestion reactor. Meanwhile, the volume ratio of the four anaerobic functional bacterial strains in the compound functional broth depends on the physicochemical parameters of the anaerobic digestion reactor, details are as follows:
1. when the ammonia concentration in the anaerobic digestion reactor is ≤ 4 g/L and the temperature is 30-43° C., the volume ratio of Coprothermobacter proteolyticus, Thermacetogenium phaeum, Methanosarcina barkeri and Methanothermobacter thermautotrophicus in the compound functional broth is 2-1:3-1:5-1:2-1;
2. when the ammonia concentration in the anaerobic digestion reactor is ≤ 4 g/L and the temperature is 50-65° C., the volume ratio of Coprothermobacter proteolyticus, Thermacetogenium phaeum, Methanosarcina barkeri and Methanothermobacter thermautotrophicus in the compound functional broth is 3-1:4-1:2-1:4-1;
3. when the ammonia concentration in the anaerobic digestion reactor is 4-7 g/L and the temperature is 30-43° C., the volume ratio of Coprothermobacter proteolyticus, Thermacetogenium phaeum, Methanosarcina barkeri and Methanothermobacter thermautotrophicus in the compound functional broth is 2-1:2-1:5-1:3-1;
4. when the ammonia concentration in the anaerobic digestion reactor is 4-7 g/L and the temperature is 50-65° C., the volume ratio of Coprothermobacter proteolyticus, Thermacetogenium phaeum, Methanosarcina barkeri and Methanothermobacter thermautotrophicus in the compound functional broth is 3-1:3-1:4-1:5-1.

In the embodiments of the present invention, the desiccated anaerobic immobilized bacterial agent is used. In order to restore the microbial activity of the desiccated anaerobic immobilized bacterial agent, the desiccated anaerobic immobilized bacterial agent needs to be activated for 12-24 h before being put into the anaerobic digestion reactor. The nutrient solution used for activation is the cultivation solution used for culturing the anaerobic functional bacterial strains when utilizing a pure bacteria culturing technique. While the same technical effect can be achieved when using gel beads prepared directly in the practical application.

In the embodiments of the present invention, the concentration unit mM is mmol/L.

In the embodiments of the present invention, OD600 is measured by spectrophotometer, the concentration of volatile suspended solids is measured referring to Water and Wastewater Monitoring and Analysis Method (the fourth edition), the concentration of ammonia is measured by Kieldahl azotometer, and the volatile organic acids is measured by gas chromatograph.

### Embodiment 1

The preparation and application of the anaerobic immobilized bacterial agent will be described in detail in the present embodiment.

At 55° C., four different anaerobic functional bacterial strains were cultured respectively utilizing a pure bacteria culturing technique to acquire corresponding culture broths with 15 of OD600. 180 mL of Coprothermobacter proteolyticus culture broth, 240 mL of Thermacetogenium phaeum culture broth, 120 mL of Methanosarcina barkeri culture broth and 240 mL Methanothermobacter thermautotrophicus culture broth were mixed to acquire a compound functional broth, then the compound functional broth was concentrated by centrifuging to acquire a functional flora precipitation, the centrifugal force was 5000 g, the centrifugal temperature was 4° C., and the centrifugal time was 5 min.

The functional flora precipitation was dissolved completely into 50 mL of 15 % (w/w) polyvinyl alcohol aqueous solution by stirring to acquire a functional flora polyvinyl alcohol solution. The functional flora polyvinyl alcohol solution was dripped into a first buffer solution containing 0.15 mol of Na₂HPO₄, 0.2 mol of NaH₂PO₄ and 50 g of H₃BO₃ per liter, standing for 24 h to generate polyvinyl alcohol gel beads. The polyvinyl alcohol gel beads were placed into a second buffer solution containing 1 mol of Na₂SO₄ per liter, standing for 48 h to generate sulfate-modified polyvinyl alcohol gel beads, the diameter of which was 0.5 cm, that was the anaerobic immobilized bacterial agent.

Four 16.3 L of anaerobic digestion reactors were chosen, the effective working volume of them was 10.5 L, operating temperature was 55° C., the concentration of volatile suspended solids was 5 g/L, pH was 6.0, the concentration of organic acids was 104 mM, and the concentration of ammonia was 1.5 g/L.

When the reactor stopped feeding, there were two groups, one was contrast group (without anaerobic immobilized bacterial agent), and another was experimental group (with anaerobic immobilized bacterial agent). At 8 days after adding the anaerobic immobilized bacterial agent, the experimental group started to restore the methanogenic ability, the maximum rate of consuming acetic acid was 15.6 mM/d. While the contrast group started to restore the methanogenic ability at 20 days after adding the anaerobic immobilized bacterial agent, the maximum rate of consuming acetic acid was 9.2 mM/d.

All the contrast group and the experimental group were two anaerobic digestion reactors, above data mentioned were the average of two sets data.

### Embodiment 2

The preparation and application of the anaerobic immobilized bacterial agent will be described in detail in the present embodiment.

At 55° C., four different anaerobic functional bacterial strains were cultured respectively utilizing a pure bacteria culturing technique to acquire corresponding culture broths with 18 of OD600. 150 mL of Coprothermobacter proteolyticus culture broth, 150 mL of Thermacetogenium phaeum culture broth, 200 mL of Methanosarcina barkeri culture broth and 250 mL Methanothermobacter thermautotrophicus culture broth were mixed to acquire a compound functional broth, then the compound functional broth was concentrated by centrifuging to acquire a functional flora precipitation, the centrifugal force was 5000 g, the centrifugal temperature was 4° C., and the centrifugal time was 5 min.

The functional flora precipitation was dissolved completely into 60 mL of 12 % (w/w) polyvinyl alcohol aqueous solution by stirring to acquire a functional flora polyvinyl alcohol solution. The functional flora polyvinyl alcohol solution was dripped into a first buffer solution containing 0.2 mol of Na₂HPO₄, 0.23 mol of NaH₂PO₄ and 55 g of H₃BO₃ per liter, standing for 24 h to generate polyvinyl alcohol gel beads. The polyvinyl alcohol gel beads were placed into a second buffer solution containing 1.5 mol of Na₂SO₄ per liter, standing for 48 h to generate sulfate-modified polyvinyl alcohol gel beads, the diameter of which was 0.8 cm, that was the anaerobic immobilized bacterial agent.

Six 8.2 L of anaerobic digestion reactors were chosen, the effective working volume of them was 5.6 L, operating temperature was 55° C., the concentration of volatile suspended solids was 3 g/L, pH was 5.5, the concentration of organic acids was 96.5 mM, and the concentration of ammonia was 4.5 g/L.

When the reactor stopped feeding, there were two groups, one was contrast group (without anaerobic immobilized bacterial agent), and another was experimental group (with anaerobic immobilized bacterial agent). At 15 days after adding the anaerobic immobilized bacterial agent, the experimental group started to restore the methanogenic ability, the maximum rate of consuming acetic acid was 9.9 mM/d. While the contrast group started to restore the methanogenic ability at 28 days after adding the anaerobic immobilized bacterial agent, the maximum rate of consuming acetic acid was 6.8 mM/d.

All the contrast group and the experimental group were three anaerobic digestion reactors, above data mentioned were the average of three sets data.

### Embodiment 3

The preparation and application of the anaerobic immobilized bacterial agent will be described in detail in the present embodiment.

At 35° C., four different anaerobic functional bacterial strains were cultured respectively utilizing a pure bacteria culturing technique to acquire corresponding culture broths with 20 of OD600. 180 mL of Coprothermobacter proteolyticus culture broth, 270 mL of Thermacetogenium phaeum culture broth, 450 mL of Methanosarcina barkeri culture broth and 180 mL Methanothermobacter thermautotrophicus culture broth were mixed to acquire a compound functional broth, then the compound functional broth was concentrated by centrifuging to acquire a functional flora precipitation, the centrifugal force was 5000 g, the centrifugal temperature was 4° C., and the centrifugal time was 5 min.

The functional flora precipitation was dissolved completely into 100 mL of 15 % (w/w) polyvinyl alcohol aqueous solution by stirring to acquire a functional flora polyvinyl alcohol solution. The functional flora polyvinyl alcohol solution was dripped into a first buffer solution containing 0.2 mol of Na₂HPO₄, 0.23 mol of NaH₂PO₄ and 60 g of H₃BO₃ per liter, standing for 24 h to generate polyvinyl alcohol gel beads. The polyvinyl alcohol gel beads were placed into a second buffer solution containing 1.2 mol of Na₂SO₄ per liter, standing for 48 h to generate sulfate-modified polyvinyl alcohol gel beads, the diameter of which was 1.0 cm, that was the anaerobic immobilized bacterial agent.

A 45.2 L of anaerobic digestion reactor was chosen, the effective working volume of them was 30.2 L, operating temperature was 35° C., the concentration of volatile suspended solids was 1 g/L, pH was 6.5, the concentration of organic acids was 50.3 mM, and the concentration of ammonia was 3.6 g/L.

When the reactor stopped feeding, there were two groups, one was contrast group (without anaerobic immobilized bacterial agent), and another was experimental group (with anaerobic immobilized bacterial agent). At 10 days after adding the anaerobic immobilized bacterial agent, the experimental group started to restore the methanogenic ability, the maximum rate of consuming acetic acid was 12.3 mM/d. While the contrast group started to restore the methanogenic ability at 15 days after adding the anaerobic immobilized bacterial agent, the maximum rate of consuming acetic acid was 7.9 mM/d.

### Embodiment 4

The preparation and application of the anaerobic immobilized bacterial agent will be described in detail in the present embodiment.

At 35° C., four different anaerobic functional bacterial strains were cultured respectively utilizing a pure bacteria culturing technique to acquire corresponding culture broths with 20 of OD600. 50 mL of Coprothermobacter proteolyticus culture broth, 50 mL of Thermacetogenium phaeum culture broth, 125 mL of Methanosarcina barkeri culture broth and 75 mL Methanothermobacter thermautotrophicus culture broth were mixed to acquire a compound functional broth, then the compound functional broth was concentrated by centrifuging to acquire a functional flora precipitation, the centrifugal force was 5000 g, the centrifugal temperature was 4° C., and the centrifugal time was 5 min.

The functional flora precipitation was dissolved completely into 20 mL of 15 % (w/w) polyvinyl alcohol aqueous solution by stirring to acquire a functional flora polyvinyl alcohol solution. The functional flora polyvinyl alcohol solution was dripped into a first buffer solution containing 0.2 mol of Na₂HPO₄, 0.23 mol of NaH₂PO₄ and 50 g of H₃BO₃ per liter, standing for 24 h to generate polyvinyl alcohol gel beads. The polyvinyl alcohol gel beads were placed into a second buffer solution containing 1.2 mol of Na₂SO₄ per liter, standing for 48 h to generate sulfate-modified polyvinyl alcohol gel beads, the diameter of which was 0.7 cm, that was the anaerobic immobilized bacterial agent.

Six 2.5 L of anaerobic digestion reactors were chosen, the effective working volume of them was 1.5 L, operating temperature was 35° C., the concentration of volatile suspended solids was 4 g/L, pH was 6.8, the concentration of organic acids was 100.6 mM, and the concentration of ammonia was 6.5 g/L.

When the reactor stopped feeding, there were two groups, one was contrast group (without anaerobic immobilized bacterial agent), and another was experimental group (with anaerobic immobilized bacterial agent). At 6 days after adding the anaerobic immobilized bacterial agent, the experimental group started to restore the methanogenic ability, the maximum rate of consuming acetic acid was 13.2 mM/d. While the contrast group started to restore the methanogenic ability at 10 days after adding the anaerobic immobilized bacterial agent, the maximum rate of consuming acetic acid was 9.8 mM/d.

All the contrast group and the experimental group were three anaerobic digestion reactors, above data mentioned were the average of three sets data.

### Effect of Embodiments

According to the preparation method and the application of anaerobic immobilized bacterial agent provided by the present invention, selecting four different anaerobic functional bacterial strains, and utilizing a pure bacteria culturing technique to acquire corresponding culture broths, then mixing the four culture broths according to a certain volume ratio to acquire a compound functional broth, subsequently concentrating the compound functional broth into a functional flora precipitation, then dissolving the functional flora precipitation into a polyvinyl alcohol aqueous solution, dripping the solution acquired into a first buffer solution to acquire polyvinyl alcohol gel beads, placing the polyvinyl alcohol gel beads into a second sulfate-containing buffer solution for modification, and the sulfate-modified polyvinyl alcohol gel beads are the anaerobic immobilized bacterial agent. The anaerobic immobilized bacterial agent is formed by the gelation of polyvinyl alcohol aqueous solution, and it is circular gel beads which are generated by wrapping gel in a thin film, the diameter of the circular gel beads is 0.5-1 cm. The anaerobic immobilized bacterial agent can be directly added into an anaerobic digestion reactor to anaerobic digestion without modifying the reactor, it can effectively relieve anaerobic digestion organic acid inhibition in situ and also can relieve organic acid accumulation under ammonia inhibition.

Coprothermobacter proteolyticus decomposes cell debris, proteins and lipids rapidly. Thermacetogenium phaeum decomposes acetic acid into carbon dioxide and hydrogen using syntrophic acetate oxidation. Methanothermobacter thermautotrophicus carries out hydrogenotrophic methanogenesis, and consumes hydrogen to create a favorable thermodynamic environment for Thermacetogenium phaeum. Methanosarcina barkeri consumes acetic acid and hydrogen by acetoclastic methanogenesis and hydrogenotrophic methanogenesis respectively. Therefore, the compound microbial community can accelerate the decrease of organic acid loading, and restore the pH of the anaerobic digestion reactor, further relieve the toxic effect of organic acid inhibition on the native microorganisms in the reactor, and finally restore the functional activity of the native microorganisms. Moreover, the adding of the compound microbial community can avoid installing organic acid separation devices, adding buffer materials and modifying the reactors, thus reducing the process cost. In addition, Coprothermobacter proteolyticus, Thermacetogenium phaeum, Methanothermobacter thermautotrophicus and Methanosarcina barkeri are all ammonia-tolerant strains, so the compound functional broth can relieve organic acid accumulation under ammonia inhibition. In conclusion, the compound functional broth is not only acid-tolerant but also ammonia-tolerant, and it has a wide applicability.

The present invention uses sulfate as a modifier, which overcomes the disadvantages where traditional polyvinyl alcohol-boric acid gel beads cannot be desiccated for preserving. The core of sulfate-boric acid has enough structural flexibility and structural strength to support the gel beads, therefore it can maintain the complete structure of the immobilized bacterial agent and the resistance to external impact.

Meanwhile, immobilizing the external compound functional bacterial agent, rather than letting them suspend in reactors in the form of free cells, can allow external bacterial agent to better colonize the native microorganisms in the reactors, and avoid the large-scale loss of external bacterial agent along with effluent, so there is no need to replenish external bacterial agent constantly. And immobilizing the external compound functional bacterial agent can also increase the cell density of the external bacterial agent, improving the treatment effect.

In addition, the modified gel beads can be mass-produced and transported over long distance because they can be desiccated for preserving, they can be used for the anaerobic digestion reactors from different geographical regions. The modified gel beads overcome the disadvantage of the common gel beads which must be prepared near the anaerobic reactors and used immediately, because the common gel beads cannot be desiccated.

In addition, the minimum dosage of the compound functional broth can be determined according to the physicochemical parameters of the anaerobic digestion reactor. Meanwhile, the mixing ratio of four different anaerobic functional bacterial strains also can be determined according to definite working conditions. Consequently, organic acid inhibition can be relived effectively and economically.

Generally, the pH of anaerobic digestion reactors is above 5. The anaerobic immobilized bacterial agent prepared by the embodiments in the present invention is appropriate for the anaerobic digestion reactors with pH > 5 (includes pH = 5). Consequently, there is no need to adjust the pH of the anaerobic digestion reactors.

Because organic acid accumulation induces organic acid inhibition, after the anaerobic immobilized bacterial agent and the reacting substrates (that is organic acid components) are mixed completely, there needs a standing reaction to degrade organic acids, and then reliving the inhibition.

The first buffer solution contains 0.15-0.2 mol of Na₂HPO₄, 0.2-0.25 mol of NaH₂PO₄ and 50-60 g of H₃BO₃ per liter, the second buffer solution contains 1-1.5 mol of Na₂SO₄ per liter. Na₂HPO₄ is benefit to maintain the steady state of microorganisms, the adding of H₃BO₃ generates boric acid cores, and Na₂SO₄ modifies the boric acid cores to acquire the modified gel beads.

When the ammonia concentration in the anaerobic digestion reactor is ≤ 4 g/L and the temperature is 30-43° C., the volume ratio of the Coprothermobacter proteolyticus, the Thermacetogenium phaeum, the Methanosarcina barkeri and the Methanothermobacter thermautotrophicus in the compound functional broth is 2-1:3-1:5-1:2-1. When the ammonia concentration in the anaerobic digestion reactor is ≤ 4 g/L and the temperature is 50-65° C., the volume ratio of the Coprothermobacter proteolyticus, the Thermacetogenium phaeum, the Methanosarcina barkeri and the Methanothermobacter thermautotrophicus in the compound functional broth is 3-1:4-1:2-1:4-1. When the ammonia concentration in the anaerobic digestion reactor is 4-7 g/L and the temperature is 30-43° C., the volume ratio of the Coprothermobacter proteolyticus, the Thermacetogenium phaeum, the Methanosarcina barkeri and the Methanothermobacter thermautotrophicus in the compound functional broth is 2-1:2-1:5-1:3-1. When the ammonia concentration in the anaerobic digestion reactor is 4-7 g/L and the temperature is 50-65° C., the volume ratio of the Coprothermobacter proteolyticus, the Thermacetogenium phaeum, the Methanosarcina barkeri and the Methanothermobacter thermautotrophicus in the compound functional broth is 3-1:3-1:4-1:5-1. These chosen ratios are determined by experimental data, the experiments simulate the four working conditions mentioned above, and there are multiple replicated reactors in every working condition. Combining with microbial sequencing analysis, the abundances of the four microorganisms are high and they are the dominant microorganisms. Therefore, the abundance ratio of the four microorganisms is chosen as the mixing volume ratio. If the volume of one microorganism is below the abundance ratio, the syntrophic relationship in the bacterial agent is destroyed, and the bacterial agent cannot relive organic acid inhibition consequently. For example, if the quantities of Methanothermobacter thermautotrophicus and Methanosarcina barkeri are low, the hydrogen produced by Coprothermobacter proteolyticus cannot be consumed, and thermodynamic environment changes, which further constructing thermodynamic barrier to Coprothermobacter proteolyticus, finally the reaction stops. If the volume of one microorganism is above the abundance ratio, the bacterial agent is wasted and the dosage of polyvinyl alcohol aqueous solution increases, so it isn’t economical. Therefore, these chosen ratios are determined according to the reaction results of the bacterial strains, it is economical and has reaction effect.

In order to restore the activity of the microorganism bacterial agent, the anaerobic immobilized bacterial agent needs to be activated for 12 h-24 h before being put into the anaerobic digestion reactor, which better reliving acid inhibition consequently.

Because microorganisms are sensitive to temperature, if the anaerobic functional bacterial strains aren’t cultured in advance at a particular temperature, the immobilized bacterial agent cannot play a role in anaerobic digestion, even the abundant microorganisms in the bacterial agent will die. For example, if the microorganisms in the bacterial agent cultured at 35° C. are added immediately into the reactor of 55° C., lag phase will be longer and the microorganisms in the bacterial agent will die, the microorganism ratio in the bacterial agent will change, consequently the bacterial agent fails. Therefore, in order to acquire the best application effect, the cultivation temperature of the anaerobic functional bacterial strains is the same with the anaerobic digesting temperature of the anaerobic immobilized bacterial agent in the anaerobic digestion reactor.

It can be known from the above that the preparation method provided by the present invention is simple, low energy consumption, and the immobilized bacterial agent acquired by this method is acid-tolerant and ammonia-tolerant. The immobilized bacterial agent can significantly improve the ability of the microorganisms to decompose the accumulated organic acids in the anaerobic digestion reactor, and there is no need to replenish the bacterial agent constantly. Compared with the prior art, the preparation method provided by the present invention has a stable effect and a low cost, and this method has no need to modify the existing reactors. The preparation method provided by the present invention can solve the problems of the loss of functional bacterial strains and the poor self-resilience of the anaerobic digestion microorganisms, and can relieve organic acid inhibition and organic acid accumulation under ammonia inhibition effectively during the treatment of organic garbage and sewage.

What is described above is merely the preferred embodiments of the present invention, and it should be noted that numerous improvements and modifications can be made by those skilled in the art without deviating from the principles of the present invention, and these improvements and modifications should also be viewed to be within the scope of the present invention.

What is claimed is:

1. A preparation method of anaerobic immobilized bacterial agent, characterized by comprising the following steps:
step 1, culturing four different anaerobic functional bacterial strains respectively at a certain temperature to acquire corresponding culture broths, then mixing four said culture broths according to a certain volume ratio to acquire a compound functional broth;
step 2, concentrating said compound functional broth to acquire a functional flora precipitation;
step 3, dissolving said functional flora precipitation into a polyvinyl alcohol aqueous solution to acquire a functional flora polyvinyl alcohol aqueous solution, then dripping said functional flora polyvinyl alcohol aqueous solution into a first buffer solution to acquire polyvinyl alcohol gel beads;
step 4, placing said polyvinyl alcohol gel beads into a second sulfate-containing buffer solution to acquire sulfate-modified polyvinyl alcohol gel beads, that is, said anaerobic immobilized bacterial agent,
wherein, said anaerobic functional bacterial strains are Coprothermobacter proteolyticus, Thermacetogenium phaeum, Methanosarcina barkeri and Methanothermobacter thermautotrophicus.

2. The preparation method of anaerobic immobilized bacterial agent according to claim 1, characterized by that
wherein, the mass percentage of polyvinyl alcohol in said polyvinyl alcohol aqueous solution in step 3 is 10-15%,
the volume ratio of said compound functional broth in step 1 to said polyvinyl alcohol aqueous solution in step 3 is 10:1-20:1.

3. The preparation method of anaerobic immobilized bacterial agent according to claim 1, characterized by that
wherein, said first buffer solution per liter contains 0.15-0.2 mol of Na₂HPO₄, 0.2-0.25 mol of NaH₂PO₄ and 50-60 g of H₃BO₃,
said second sulfate-containing buffer solution per liter contains 1-1.5 mol of Na₂SO₄,
the diameter of said anaerobic immobilized bacterial agent is 0.5-1 cm.

4. An anaerobic immobilized bacterial agent, characterized by being made by the preparation method of anaerobic immobilized bacterial agent according to claim 1, wherein said anaerobic immobilized bacterial agent is circular gel beads which are generated by wrapping gel in a thin film, and the diameter of said circular gel beads is 0.5-1 cm.

5. An application of the anaerobic immobilized bacterial agent according to claim 4 for anaerobic digestion.

6. The application of the anaerobic immobilized bacterial agent for anaerobic digestion according to claim 5, characterized by that
wherein, utilizing a pure bacteria culturing technique, culturing four anaerobic functional bacterial strains respectively to acquire corresponding culture broths at a certain temperature, OD600 of said culture broths is 15-20, then mixing different said culture broths according to a certain volume ratio to acquire a compound functional broth,
specific operation of said anaerobic digestion is that adding said anaerobic immobilized bacterial agent into an anaerobic digestion reactor to anaerobic digestion, the cultivation temperature of said anaerobic functional bacterial strains is the same with the anaerobic digesting temperature of said anaerobic immobilized bacterial agent in said anaerobic digestion reactor,
four said anaerobic functional bacterial strains are Coprothermobacter proteolyticus, Thermacetogenium phaeum, Methanosarcina barkeri and Methanothermobacter thermautotrophicus respectively.

7. The application of the anaerobic immobilized bacterial agent for anaerobic digestion according to claim 6, characterized by that
wherein, the minimum dosage of said compound functional broth is calculated by the following formula:
$${V}_{0} = \frac{{C}_{t o t a l} \times {10}^{- \text{pH}} \times {C}_{v s} \times {V}_{1}}{0.0072}$$
V₀ is the minimum dosage of said compound functional broth, L,
Cₜₒₜₐₗ is the total concentration of organic acids in the anaerobic digestion reactor, in terms of acetate, mM,
pH is the pH in the anaerobic digestion reactor,
Cᵥₛ is the concentration of volatile suspended solids in the anaerobic digestion reactor, g/L,
V₁ is the effective working volume of the anaerobic digestion reactor, L.

8. The application of the anaerobic immobilized bacterial agent for anaerobic digestion according to claim 6, characterized by that
wherein, when the ammonia concentration in said anaerobic digestion reactor is ≤ 4 g/L and the temperature is 30-43° C., the volume ratio of said Coprothermobacter proteolyticus, said Thermacetogenium phaeum, said Methanosarcina barkeri and said Methanothermobacter thermautotrophicus in said compound functional broth is 2-1:3-1:5-1:2-1;
when the ammonia concentration in said anaerobic digestion reactor is ≤ 4 g/L and the temperature is 50-65° C., the volume ratio of said Coprothermobacter proteolyticus, said Thermacetogenium phaeum, said Methanosarcina barkeri and said Methanothermobacter thermautotrophicus in said compound functional broth is 3-1:4-1:2-1:4-1.

9. The application of the anaerobic immobilized bacterial agent for anaerobic digestion according to claim 6, characterized by that
wherein, when the ammonia concentration in said anaerobic digestion reactor is 4-7 g/L and the temperature is 30-43° C., the volume ratio of said Coprothermobacter proteolyticus, said Thermacetogenium phaeum, said Methanosarcina barkeri and said Methanothermobacter thermautotrophicus in said compound functional broth is 2-1:2-1:5-1:3-1;
when the ammonia concentration in said anaerobic digestion reactor is 4-7 g/L and the temperature is 50-65° C., the volume ratio of said Coprothermobacter proteolyticus, said Thermacetogenium phaeum, said Methanosarcina barkeri and said Methanothermobacter thermautotrophicus in said compound functional broth is 3-1:3-1:4-1:5-1.

10. The application of the anaerobic immobilized bacterial agent for anaerobic digestion according to claim 6, characterized by that
wherein, desiccating said anaerobic immobilized bacterial agent to acquired desiccated gel beads, and activating said desiccated gel beads for 12-24 h before adding them into said anaerobic digestion reactor, the nutrient solution used for activation is said cultivation solution used for culturing said anaerobic functional bacterial strains when utilizing a pure bacteria culturing technique.
